(19) United States
(12) Patent Application Publication
(10) Pub. No.: US 2009/0171173 A1
(43) Pub. Date: Jul. 2, 2009

(54) SYSTEM AND METHOD FOR REDUCING MOTION ARTIFACTS IN A SENSOR

(75) Inventors: Clark R. Baker, JR., Newman, CA (US); Carine Hoarau, Lafayette, CA (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC, Boulder, CO (US)

(21) Appl. No.: 12/340,955

(22) Filed: Dec. 22, 2008

Related U.S. Application Data
(60) Provisional application No. 61/009,679, filed on Dec. 31, 2007.

(51) Int. Cl. A61B 5/1455; A61B 5/00

(52) U.S. Cl. 600/324; 600/310; 600/300

(57) ABSTRACT

Embodiments disclosed herein may include a patient sensor which has a low-friction exterior coating. In an embodiment, the exterior surface of the sensor may come into contact with external items, such as, for example, bed linens, clothing, unintended parts of the patient's body, or other people. The low-friction coating disposed on the exterior of the sensor may include a material having a relatively low coefficient of friction with respect to these external items. In an embodiment, the low-friction material may include, for example, a fluoropolymer, a polypropylene, or a polyethylene. Additionally, in an embodiment, an internal surface of the sensor that is in contact with the patient may have a relatively high-friction coating, such as an adhesive. In an embodiment, a stack of adhesive layers may be disposed on the internal surface around one or more light emitting and/or detecting optics.

### RELATED APPLICATION

This application claims priority from U.S. Provisional Application No. 61/009,679, filed, Dec. 31, 2007, which is hereby incorporated by reference herein in its entirety.

### BACKGROUND

The present disclosure relates generally to medical devices and, more particularly, to sensors used for sensing physiological parameters of a patient.

This section is intended to introduce the reader to various aspects of art that may be related to various aspects of disclosed embodiments, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of healthcare, caregivers (e.g., doctors and other healthcare professionals) may often desire to monitor certain physiological characteristics of their patients. Accordingly, a wide variety of monitoring devices have been developed for monitoring many such physiological characteristics. These monitoring devices often provide doctors and other healthcare personnel with information that facilitates provision of the best possible healthcare for their patients. As a result, such monitoring devices have become a perennial feature of modern medicine.

One technique for monitoring physiological characteristics of a patient is commonly referred to as pulse oximetry, and the devices built based upon pulse oximetry techniques are commonly referred to as pulse oximeters. Pulse oximeters may be used to measure and monitor various blood flow characteristics of a patient. For example, a pulse oximeter may be utilized to monitor the blood oxygen saturation of hemoglobin in arterial blood (SpO₂), the volume of individual blood pulsations supplying the tissue, and/or the rate of blood pulsations corresponding to each heartbeat of a patient. In fact, the “pulse” in pulse oximetry refers to the time-varying amount of arterial blood in the tissue during each cardiac cycle.

Pulse oximeters may utilize a non-invasive sensor that is placed on or against a patient's tissue that is well perfused with blood, such as a patient's finger, toe, forehead or earlobe. The pulse oximeter sensor emits light and photoelectrically senses the light after passage through the perfused tissue. Thus, the sensed light may be utilized to determine the absorption and/or scattering of the light through the tissue.

The light emitted by the sensor may be selected to include one or more wavelengths that are absorbed or scattered in an amount related to the presence of oxygenated versus de-oxygenated hemoglobin in the blood. Thus, data collected by the sensor relating to detected light may be used to calculate one or more of the above-referenced physiological characteristics based upon the absorption or scattering of the light. For example, a determination of the amount of light absorbed and/or scattered may be used to estimate an amount of oxygen in the tissue using various algorithms.

Pulse oximetry monitors may measure pulsatile, dynamic changes in the amount and type of blood constituents in tissue based on corresponding changes between light emitted and detected by the sensor. However, other events besides the pulsing of arterial blood may lead to modulation of the light path, direction, and the amount of light detected by the sensor. These other events may result in measurement errors. Indeed, pulse oximetry may be sensitive to movement, and various types of motion may cause artifacts that may obscure a blood constituent signal.

A wide variety of sources of motion resulting in motion artifacts may be found in emergency room, critical care, intensive care, and trauma center settings, where pulse oximetry is commonly used for patient monitoring, For example, sources of motion artifact in these settings may include moving of a patient or the sensor by healthcare workers, physical motion of an unanaesthetised or ambulatory patient, partial opening of a clip in response to patient motion, shivering, seizures, agitation) response to pain and loss of neural control. These motions oftentimes have similar frequency content to a pulse) and may lead to similar or even larger optical modulations than the pulse.

### SUMMARY

Certain aspects commensurate in scope with this disclosure are set forth below. It should be understood that these aspects are presented merely to provide the reader with a brief summary of certain embodiments the disclosure might take and that these aspects are not intended to limit the scope of the disclosure. Indeed, the invention may encompass a variety of aspects that may not be set forth below.

According to an embodiment, there may be provided a sensor, including a sensor body and a low-friction coating on an exterior surface of the sensor body. The exterior surface may be placed away from a patient's tissue, and the low-friction coating may be made of a material having a coefficient of static friction of less than one (1) with respect to cotton, linen, hemp, and/or combinations thereof.

In accordance with an embodiment, there may be provided a sensor, including a bandage-style sensor body for affixing the sensor to a patient) an interior coating for adhesion of the bandage-style sensor body to the patient, and an exterior coating made of a low-function material.

According to an embodiment, there may be provided a sensor, including a low-friction coating on an exterior surface of a sensor body, an emitter configured to emit light into tissue of the patient during operation; and a detector configured to detect the light.

In accordance with an embodiment, there may be provided a method, including providing a bandage-style sensor having an emitter and a detector installed therein and coating an exterior surface of the bandage-style sensor with a material having a coefficient of static friction of less than one (1) with respect to cotton, linen, hemp, and/or combinations thereof.

Finally, according to an embodiment, there may be provided a method, including providing a sheet of sensor body material, applying a low-friction coating to a first surface of the sheet, dividing the sheet into individual sensor bodies, and coupling the sensor body to light emission and detection optics.

### BRIEF DESCRIPTION OF THE DRAWINGS

Advantages of this disclosure may become apparent upon reading the following detailed description and upon reference to the drawings in which:

FIG. 1 is a perspective view of a pulse oximeter coupled to a multi-parameter patient monitor and a sensor in accordance with aspects of an embodiment; and

FIGS. 2 and 3 are cross-sections of the various embodiments of the sensor applied to a patient's digit in accordance with aspects of embodiments.

### DETAILED DESCRIPTION OF SPECIFIC EMBODIMENTS

Various embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Patient monitoring sensors may be susceptible to interference due to movement relative to the patient's tissue. For example, with a traditional sensor, such as a bandage sensor, the exterior of the sensor may tend to snag on the patient's bed linens, clothing, skin, or hair due to friction between the sensor and the cloth, resulting in motion artifacts impacting the calculation of the patient's physiological parameters. Accordingly, the present embodiments may provide a patient sensor having a low-friction exterior and a high-friction or tacky coupling portion configured to be securely affixed to the patient's tissue. That is, the exterior surfaces (i.e., surfaces of the sensor that may be exposed to the environment when the sensor is applied to the patient) may include a low-friction material configured to resist external influences that would tend to move the sensor relative to the patient's tissue. A low-friction material may be defined as a material that has a coefficient of friction of less than one relative to common items used in medical facilities, such as bed linens and clothing, Exemplary low-function materials that may be utilized in accordance with present embodiments include a fluoropolymer, a polyethylene, a polypropylene, and so forth. For example, a polyethylene such as Tyvek® may be a desirable coating due to its biocompatibility and ability to be sterilized. Similarly, Teflon® exhibits a very low coefficient of friction relative to many other materials. Medical facilities may utilize natural cloths in bed linens and gowns, such as, for example, cotton, linen, hemp, and so forth. Additionally, present embodiments may include a high-friction or tacky interior which promotes stationary adhesion of the sensor to the patient.

Embodiments are directed to reducing interference due to motion of a sensor relative to a patient in calculating the patient's physiological parameters. Specifically, present embodiments are directed to avoiding or limiting errors in the calculation of pulse rate, blood oxygen saturation (SpO₂), tissue water percentage, and so forth, that result from motion artifacts. For example, motion artifacts may be caused by moving a sensor in relation to the tissue, by increasing or decreasing the physical distance between emitters and detectors in a sensor, by changing the direction of emitters or detectors with respect to tissue or each other, by changing the angles of incidence and interfaces probed by the light, by directing the optical path through different amounts or types of tissue, or by expanding, compressing or otherwise altering tissue near a sensor. Motion artifacts may be reduced in accordance with present embodiments by limiting the impact of items in a patient's surroundings (e.g., bed sheets, hospital gowns, other body parts, etc.) on the relative position of a sensor coupled to the patient.

A sensor in accordance with various embodiments may be formed of various types of material, including both low-friction material and high-friction material (e.g., adhesive). The low-friction material is included on exterior surfaces of the sensor that are not expected to be in contact with the patient's tissue. For example, the sensor may be made of a low-friction material and may include a high-friction coating on surfaces which are in contact with the patient's tissue during operation. In addition, the sensor may be made of a relatively high-friction material and coated with the low-friction material. Furthermore, the sensor may be coated in some areas with high-function material and in other places with the low-function material.

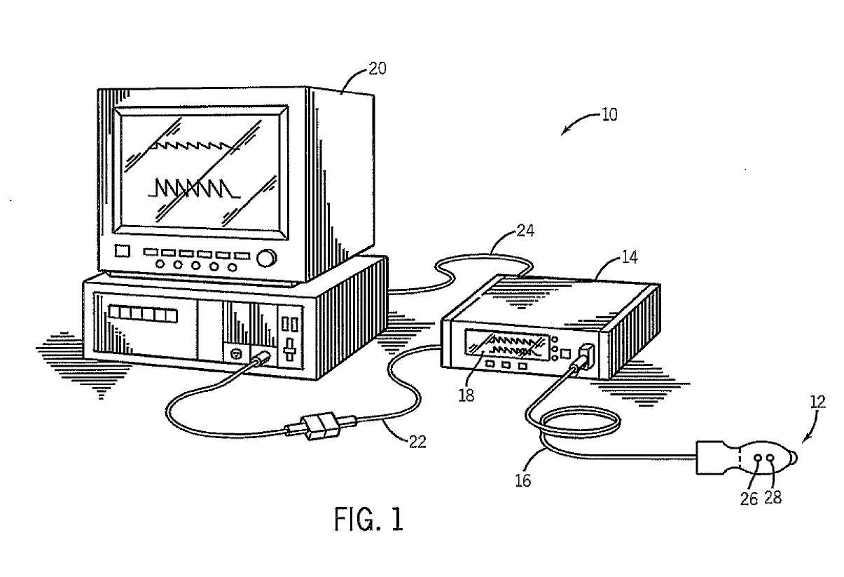

Prior to discussing embodiments of such sensors in detail, it should be appreciated that although the embodiments introduced above and discussed in detail below may be implemented for a variety of medical devices, the present disclosure discusses the implementation of these embodiments in a pulse oximetry system. FIG. 1 is a perspective view of such a pulse oximetry system 10 in accordance with an embodiment. Sensor 12 may be used in conjunction with a monitor 14. In the depicted embodiment, a cable 16 connects the sensor 12 to the monitor 14. The sensor 12 and/or the cable 16 may include or incorporate one or more integrated circuit devices or electrical devices, such as a memory, processor chip, or resistor, that may facilitate or enhance communication between the sensor 12 and the monitor 14. Likewise, the cable 16 may be an adaptor cable, with or without an integrated circuit or electrical device, for facilitating communication between the sensor 12 and various types of monitors, including older or newer versions of the monitor 14 or other physiological monitors. In other embodiments, the sensor 12 and the monitor 14 may communicate via wireless features, such as using radio, infrared, or optical signals. In such embodiments, a transmission device (not shown) may be connected to the sensor 12 to facilitate wireless transmission between the sensor 12 and the monitor 14. The cable 16 (or corresponding wireless transmission) is typically used to transmit control or timing signals from the monitor 14 to the sensor 12 and/or to transmit acquired data from the sensor 12 to the monitor 14. In another embodiment, the cable 16 may be an optical fiber that enables optical signals to be conducted between the monitor 14 and the sensor 12.

In an embodiment, the monitor 14 may be a pulse oximeter; such as those available from Nellcor Puritan Bennett LLC, or may be a monitor for measuring other body fluid related metrics using spectrophotometric or other techniques. Additionally, the monitor 14 may be a multi-purpose monitor suitable for performing pulse oximetry and/or measurement of tissue water fraction, or other combinations of physiological and/or biochemical monitoring processes, using data acquired via the sensor 12. Measured physiological parameters may be displayed on a display 18. Furthermore, the monitor 14 may be coupled to a multi-parameter monitor 20 via a cable 22 connected to a sensor input port and/or via a cable 24 connected to a digital communication port.

In an embodiment, the sensor 12 includes an emitter 26 and a detector 28 which may be of any suitable type. For example, the emitter 26 may be one or more light emitting diodes adapted to transmit one or more wavelengths of light, such as in the red to infrared range, and the detector 28 may be a photodetector, such as a silicon photodiode package, selected to receive light in the range emitted from the emitter 26. In each of the embodiments discussed herein, it should be understood that the locations of the emitter 26 and the detector 28 may be exchanged. In either arrangement, the sensor 12 will perform in substantially the same manner.

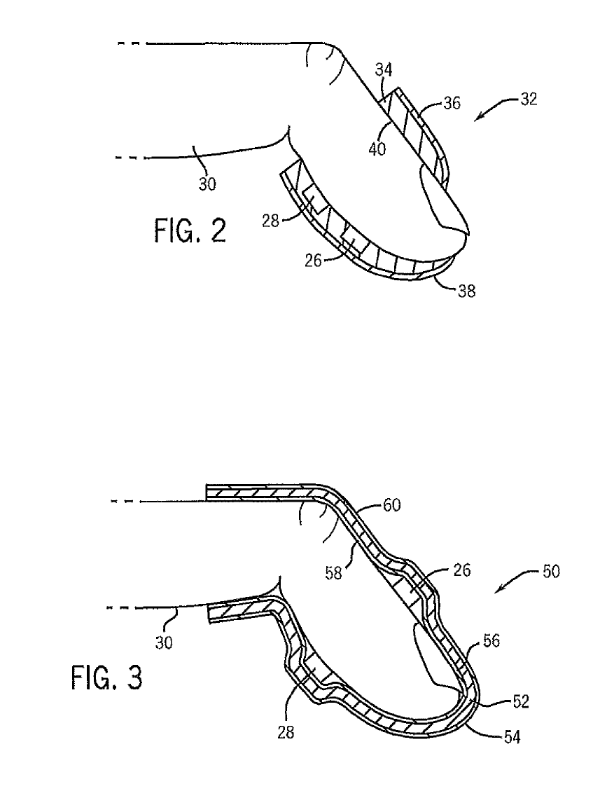

In an embodiment, sensors 12 for use with the described monitor 14 may be applied to a patient's tissue, such as a digit, an earlobe, a heel, a forehead, or any other appropriate area for measuring physiological parameters. As described in more detail below, the sensor 12 may have a low friction material on surfaces which are not designed to be in contact with the patient's tissue or which are expected to be in contact with external items. For example, the exterior of the sensor 12 may often contact the patient's bed linens or clothing. A low friction exterior may reduce the likelihood that the sensor 12 will snag on such items, thereby reducing movement of the sensor 12 in relation to the patient's tissue. Examples of such sensors are illustrated in FIGS. 2-3. It should be noted that while the sensors introduced above and discussed in detail below may be applied to different tissue sites, the present disclosure will discuss the application of these sensors to a patient's digit 30.

An embodiment of a sensor 32 is illustrated in FIG. 2 as being coupled to the patient's digit 30. Specifically, in the illustrated embodiment, a sensor body 34, such as a bandage sensor or a clip sensor, secures the emitter 26 and the detector 28 to the patient's digit 30. In addition, a low-friction coating 36, such as, for example, a fluoropolymer, a polypropylene, or a polyethylene, has been applied to the sensor body 34. For example, the low-friction coating 36 may cover an exterior surface 38 of the sensor body 34. The low-friction coating 36 may have a coefficient of static friction (μₛ) and/or a coefficient of kinetic friction (μₖ) of less than one with respect to common cloths that may be found in medical facilities, such as cotton, linen, hemp, and so forth. The coefficients of friction may be measured, for example, in accordance with ASTM G115-04, “Standard Guide for Measuring and Reporting Friction Coefficients.” In an embodiment, the coefficient of static friction between the low-friction coating 36 and the commonly used cloth may be determined by stacking the materials together on a flat surface and increasing the incline of the surface until the materials slide with respect to one another. In this embodiment, the coefficient of static friction may be determined according to the equation μₛ=tan(θ) where θ is the angle of the incline at which the materials begin to slide.

In an embodiment, an interior patient contact surface 40 may be free of the low-friction coating 36 to promote adhesion to the patient's digit 30. The interior patient contact surface 40 may include an adhesive, as described below, or the sensor 32 may be secured to the patient via alternative means, such as, for example, the SoftCare® nonadhesive sensors available from Nellcor Puritan Bennet LLC and/or Covidien. Accordingly, the sensor 32 is configured such that it will not move easily relative to the patient hut may move easily relative to external objects, such as bed linens or clothing, or parts of the patient's body that the sensor is not intended to contact.

An embodiment of a sensor 50 is illustrated in FIG. 3. In this embodiment, the sensor 50 may include a sensor body 52 having both a low-friction exterior coating 54 and a high-friction interior coating 56. In an embodiment, the sensor body 52 may be a bandage sensor, such as the OxiMax® sensors available from Nellcor Puritan Bennett LLC and/or Covidien. The high-friction coating 56 may be applied to an interior surface 58 of the sensor body 52 may promote stability of the emitter 26 and the detector 28 in relation to the patient's digit 30. That is, the coating 56 may enable the sensor 50 to adhere to the patient. In certain embodiments, the adhesive coating 56 may include a stack of adhesive layers. After one or more uses, a top adhesive layer may be removed to expose a new adhesive layer, thereby providing adhesion even after the sensor 50 is removed and reapplied multiple times.

In addition, an exterior surface 60 of the sensor body 52 may be coated with the low-friction material 54, such as, for example, a fluoropolymer, a polypropylene, or a polyethylene, to reduce interference with external objects. As described above, the coefficient of static and/or kinetic friction of the low-friction coating 54 with respect to common cloths may be less than one.

Embodiments of sensors described above (e.g., 32 and/or 50) may be manufactured via various processes. For example, the sensor body (e.g., 34 and/or 52) may be molded, and the low-function coating (e.g., 36 and/or 54) and/or the high-friction coating (e.g., 46 and/or 56) may be applied by a spray coating. In another embodiment, a sheet of sensor body material may be produced, and the low-friction coating (e.g., 36 and/or 54) and/or the high-friction coating (e.g., 46 and/or 56) may be painted onto the sheet. The sensor body (e.g., 34 and/or 52) may then be cut out of the sheet. It should be understood that other manufacturing methods may be utilized to produce the sensors described herein and their equivalents.

While the subject of this disclosure may be susceptible to various modifications and alternative forms, specific embodiments have been shown by way of example in the drawings and have been described in detail herein. However, it should be understood that this disclosure is not intended to be limited to the particular forms disclosed. Rather, the invention is to cover all modifications, equivalents, and alternatives falling within the spirit and scope of the invention as defined by the following appended claims.

What is claimed is:

1. A sensor, comprising:
a sensor body; and
a generally low-friction coating on an exterior surface of the sensor body;
wherein the exterior surface is configured to be placed away from a tissue of a patient, and wherein the low-friction coating comprises a material having a coefficient of static friction of generally less than one (1) with respect to cotton, linen, and/or hemp, and/or combinations thereof.

2. The sensor of claim 1, comprising a high-function coating disposed on an interior surface of the sensor body, wherein the interior surface is configured to be placed on and/or adjacent the tissue of the patient.

3. The sensor of claim 2, wherein the high-friction material comprises a stack of adhesive layers.

4. The sensor of claim 1, wherein the low-friction coating comprises fluoropolymer, polypropylene, and/or polyethylene, and/or combinations thereof.

5. The sensor of claim, wherein the sensor body comprises a bandage-style sensor body.

6. The sensor of claim 1, wherein the low-friction material comprises a fluoropolymer, polypropylene, and/or polyethylene.

7. The sensor of claim 1, wherein the low-friction material comprises a polyethylene.

8. The sensor of claim 1, wherein the low-friction material comprises a material having a coefficient of static friction of generally less than one (1) with respect to cotton, linen, and/or hemp, and/or combinations thereof.

9. A sensor, comprising:
a low-friction coating on an exterior surface of a sensor body;
an emitter configured to emit light into tissue of the patient during operation; and
a detector configured to detect the light.

10. The sensor of claim 9, wherein the sensor comprises a high-friction coating on an interior surface of the sensor body.

11. The sensor of claim 9, wherein the sensor comprises a stack of adhesive layers disposed on an interior surface of the sensor body generally adjacent to the emitter and/or the detector.

12. The sensor of claim 9, comprising a monitor configured to receive signals from the sensor and to calculate a physiological parameter of the patient based at least in part upon the received signals.

13. The sensor of claim 12, wherein the physiological parameter comprises pulse rate, blood oxygen saturation, and/or tissue water percentage, and/or combinations thereof.

14. The sensor of claim 9, wherein the low friction coating comprises fluoropolymer, polypropylene, and/or polyethylene, and/or combinations thereof.

15. The sensor of claim 9, wherein the low friction coating comprises a material having a coefficient of static friction of generally less than one (1) with respect to cotton, linen, and/or hemp, and/or combinations thereof.

16. A method, comprising:
providing a bandage-style sensor comprising an emitter and a detector installed therein; and
coating an exterior surface of the bandage-style sensor with a material having a coefficient of static friction of generally less than one (1) with respect to cotton, linen, and/or hemp, and/or combinations thereof.

17. The method of claim 16, comprising coating an interior surface of the bandage-style sensor with a high-friction material.

18. The method of claim 16, comprising applying a stack of adhesive layers to an interior surface of the bandage-style sensor adjacent the emitter and/or the detector.
